(19) United States
(12) Patent Application Publication
(10) Pub. No.: US 2022/0151937 A1
(43) Pub. Date: May 19, 2022

(54) COMPOSITION FOR NAP PROMOTION

(71) Applicant: NapJitsu, Inc., Huntington Beach, CA (US)

(72) Inventor: John Ferrari, Newport Beach, CA (US)

(21) Appl. No.: 17/529,160

(22) Filed: Nov. 17, 2021

Related U.S. Application Data
(60) Provisional application No. 63/115,373, filed on Nov. 18, 2020.

(51) Int. Cl. A61K 9/24; A61K 9/48; A61P 25/20

(52) U.S. Cl. CPC A61K 9/209; A61P 25/20; A61K 9/4891

(57) ABSTRACT

The present invention relates to an oral formulation for promoting napping in a subject. The formulation may comprise an outer layer comprising a sedative and/or relaxant, and an inner layer comprising a stimulant.

### CROSS-REFERENCE TO RELATED APPLICATIONS

This application claims the benefit of U.S. Provisional Application 63/115,373 filed on Nov. 18, 2020, the disclosure of which is incorporated herein by reference.

### FIELD OF THE INVENTION

The present invention relates to compositions for promoting restorative napping and methods for using the compositions.

### BACKGROUND OF THE INVENTION

Many Americans today are not getting the recommended amount of sleep each night. Americans report an average of 7 hours, 36 minutes/night; 40 minutes longer on non-workdays. One-third, however, get less than recommended amount (<7 hours). According to the American Sleep Association, every year approximately 40 million Americans, if not more, are afflicted by chronic, long-term sleep disorders. Spending related to sleep has increased 8.8% annually since 2008, reaching about $32 billion in 2012. In 2012, 73% of American Internet users went online to research health information, and 43% looked specifically for sleep remedies. Problems associated with sleep deficiencies extend well beyond fatigue and crankiness. Recent sleep studies have linked insufficient sleep to a host of problems including hypertension, depression, anxiety, diabetes, improper immune functioning, forgetfulness, clumsiness, jumpiness and even things like teen sports injuries. Forty-five percent of adults surveyed by the National Sleep Foundation reported that they “rarely” or “never” get a good night's sleep in any given week. This has resulted in consumers compensating either by ingesting sleep aids to help them fall asleep and stay asleep for eight hours through the night and/or consuming large amounts of caffeine to stay awake at work or at school.

The OTC sleep-aid market has continued to grow in recent years, hitting $759MM in sales in 2013. The category is divided into drug and supplement ingredients: most commonly, diphenhydramine (an antihistamine) and melatonin, respectively. Diphenhydramine sleep aids are known to help produce long, drug-induced periods of sleep that typically last between 6-8 hours. Additionally, antihistamine-based sleep aids often have a paradoxical effect, especially for those older than 65, and can cause wakefulness. Melatonin in common doses found over-the-counter such as 3 mg-10 mg are also often consumed to help users get a full night of sleep. All existing sleep aids can cause drowsiness that can last between three and eight hours and cannot be taken after 4 AM for those needing to get up for work in the morning. For example, the directions for melatonin-based^{MidNite®} state: “Do not drive or operate equipment for three hours after taking.”

Americans are also consuming caffeine in record numbers, and the energy category has exploded in recent years. Globally, the energy drinks and shots business market grew into a $27.5 billion business last year from $3.8 billion in 1999 and U.S. Sales of energy drinks and shots in 2013 totaled about $9B—up 17% versus 2012. Energy shots are expected to continue to grow, even though 59% of energy drink users are concerned about safety. 95% of adults drink at least one caffeinated beverage a day and average 3.1 beverages per workday. Simply caffeinating a tired brain can actually decrease memory performance and can make one feel more wired, while also making one more prone to make mistakes.

Sleep-deprived Americans continue to compensate for poor sleep with naps. Forty-two percent of adults 18-64 report napping at least once a week, with an average of 1.4 naps taken during the workweek and one nap on non-work-days. Napping has plenty of proven benefits including, relaxation, reduced fatigue, increased alertness, improved mood, improved mental performance, enhanced creativity, improved reaction, better memory, less confusion, and fewer accidents and mistakes. The recommended length of nap is less than 45 minutes. A nap between 15 and 30 minutes is sometimes called a “power nap” and is recommended for short-term alertness and energy, improving alertness and performance, and helping the subject feel more alert and less sleepy even if they didn't sleep much the previous night. About one-third of us nap on any given day. A majority of us nap at least once per month. Short naps do not interfere with nighttime sleep. Many adults may find it difficult to fall asleep quickly enough during the day to achieve the described power nap in their allotted nap schedule. Even among those whose schedules can accommodate a longer nap, many are reluctant because they fear the “sleep inertia” or grogginess that can set in after naps.

Napping with caffeine is useful. While not wishing to be constrained by any present theory, it is believed that the caffeine-nap nexus is grounded in the science of the electro-chemical physiology of the human brain. Specifically, we have found that a properly timed dose of coffee can trick the brain into replacing the naturally occurring brain chemical adenosine, a sleep enabler, with caffeine. The net effect is that caffeine consumption immediately followed by a 20-30 minute nap will provide two key benefits:
The benefit of sleep itself
The kick of caffeine upon awakening.
   So 20-30 minutes after the combined benefits of the caffeine-nap, individuals feel alert, focused, and energized—an effect that continues to increase steadily over a period of at least five hours. Other science has repeatedly shown the benefits of caffeine and naps for the consolidation of memory—the process by which short term memory is cemented in the brain as long-term memory.

### Overview of the Invention

Provided herein are oral formulations to help create a better power nap. One preferred embodiment of the present invention is a composition that will generally lead to a restorative nap of 30 minutes. Another preferred embodiment is directed to generating a restorative nap in about 10 minutes. One preferred embodiment uses multiple pills with some pills containing a stimulant. By varying the release profile and the substance, it is possible to tailor the nap to the person and to the environment or duration of the nap. We have found that with an embodiment we designate as nootropic Nap 30, seven subjective metrics including tiredness, energy, focus, alertness, feeling good, memory and mood improved over a five-hour period. Compared with coffee or placebo, this Nap 30 composition was more consistent in its effects over time and more stable. Lower in caffeine than coffee, no harmful effects were reported, but 56% did report overall feelings of improved energy, focus and alertness five hours after taking just two pills. Embodiments include a tablet for the sleep ingredients wherein the tablets dissolve faster in the consumer's mouth providing almost instant effects. Embodiments can include a time release caffeine capsule that begins working approximately 10-30 minutes after consumption. Embodiments also include nootropics to boost energy and focus levels as high as possible throughout the day. In certain embodiments the formulation referred to as Nap10 are preferred. Nap10 removes the double tablet formulation and removes any sleep inducing ingredients for a ten minute nap. Nap10 also lacks a heavy initial doses of caffeine to avoid producing an over-caffeinated and jittery effect. In some embodiments, a multiple layer caffeine release system allows for multiple smaller doses of caffeine to be released into the body over a time period. In some embodiments, there are 3 time release layers of about 50 mg of caffeine in each to produce a period of about 3 hours of focused and alert feeling. Some embodiment produce an experience of caffeine that lasting about 5-7 hours. Some embodiments include doses of nootropics and mushrooms to really boost energy and focus.

Another embodiment is a simple pill comprising an outer layer, which may comprise a sedative and optionally a relaxant, and an inner layer comprising a stimulant. The sedative, relaxant, and stimulant for one pill or the outer pill coating may be from a natural source. For a single pill embodiment the inner layer may comprise a capsule and the outer layer may comprise a coating.

The sedative in some embodiment compositions may comprise melatonin, valerian root extract, hops extract, kava extract, gamma aminobutyric acid (GABA), MCT oil, fractionated coconut oil, lemon balm leaves powder, lavender flower powder, L-tryptophan, passion flower powder, or magnesium citrate. The relaxant in some embodiment compositions may comprise chamomile extract, lavender extract, lemon balm extract, or passion flower extract. The stimulant in some embodiment compositions may comprise caffeine, which may be from a green tea extract, guarana extract, coffee extract, or yerba mate extract, lemon argentina, copaiba, orange sweet oil, or grapefruit pink. The nootropics in some embodiment compositions may comprise niacinamise, pyridoxal 5 phosphate, methyltetrahydrofolate, methylcobalmin, calcium d-pantothenate, alpha-lipoic acid, L-tyrosine, L-glutamine, white willow bark powder, DMAE bitartrate, acetyl L-carnitine, or ginkgo leaves powder.

Embodiments can also include a coated capsule comprising a liquid fill, a shell surrounding the liquid fill, and a coating around the shell. The liquid fill may optionally comprise the stimulant, a liquid vehicle, a plasticizer, and water. The shell may comprise gelatin, an opacifier, optionally a colorant, and optionally water. The coating may comprise a cellulose derivative, a polyether, a mineral oil, the sedative, and the relaxant. The liquid vehicle may comprise a vegetable oil, and the plasticizer may comprise glycerol. The opacifier may comprise titanium dioxide. The cellulose derivative may comprise hypromellose and the polyether may comprise PEG 400. The liquid fill may comprise 500 mg green tea extract, 300 mg vegetable oil, 50 mg glycerol, and water. The shell may comprise 265 mg gelatin, 10 mg titanium dioxide, and 1 mg colorant. The coating may comprise 52 mg hypromellose, 28 mg PEG 400, 6 mg mineral oil, 1 mg melatonin, and 1 mg chamomile extract.

Further provided herein is a method of promoting napping in a subject, which comprises administering the oral formulation or the coated capsule to a subject in need thereof.

### DETAILED DESCRIPTION

The inventors have discovered that oral formulations comprising multiple pills or a single controlled release pill with an outer portion comprising a sedative and/or relaxant, and an inner portion comprising a stimulant, are surprisingly effective in promoting napping in a subject for about 10-45 minutes, with no nap hangover or grogginess. In a preferred embodiment, this time period is about 30 minutes. When the formulations are swallowed by the subject, the first pill of an outer portion of a single pill may dissolve in the mouth or stomach, where a released sedative and/or relaxant may aid in relaxation and restful sleep. About 20 to 30 minutes after the formulation has been swallowed, the inner portion may dissolve, releasing the stimulant into the stomach. The stimulant may be absorbed from the stomach and have a pharmacological effect within about 30-60 minutes after the formulation is consumed. The formulation may enhance the nap experience of the subject by improving the quality of sleep, while delivering an energy boost as the subject awakens.

One pill may help the subject relax to begin their nap and to help improve the quality of their nap; another pill then releases a time-delayed kick of a natural stimulant to help clear the mind 30-45 minutes later when the subject is awakened. The subject then awakes refreshed and not groggy while still enjoying the proven benefits of a short nap. Nap30 and Nap10 are non-addicting and designed for improving short-duration naps that are more restful for the brain than purely consuming caffeine, while avoiding the 3-8 hours of grogginess that can ensue from traditional sleep aids. In some embodiments the components can be put into a single pill with timed release of the relaxant and stimulant or with the coating of the pill forming the initial drug release profile and the inner portion of the pill forming the latter drug release profile.

### 1. Definitions

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### 2. Nap-Promoting Formulation

Provided here is a formulation for promoting napping in a subject in need of rest or mental clarity. The napping may not induce grogginess. The formulation may be an oral formulation, and may comprise multiple pills or a single pill with an inner portion and an outer portion. The one pill or the inner portion of some embodiments may comprise a stimulant and the outer portion of a pill of some embodiments may comprise a sedative and/or a relaxant. The first pill or outer portion of a pill may begin dissolving in the subject's mouth and release the sedative and/or relaxant, thereby assisting the subject in achieving restful sleep within about 10 to 15 minutes of consuming the formulation. The second pill or inner portion may dissolve in the subject's stomach within about 20 to 30 minutes, which may release the stimulant. The stimulant may have a pharmacological effect within 10 minutes of being absorbed in the subject's stomach, thereby causing the subject to feel energized. The formulation may thus promote a nap of about 10-45 minutes in the subject.

a. Stimulant

The stimulant may comprise caffeine, which may comprise a caffeine from a natural source. The source of the caffeine may be a green tea extract, guarana extract, coffee extract, or yerba mate extract, or a combination thereof. While the stimulant may comprise caffeine in particular, the stimulant may also comprise, or comprise an extract of, Acetyl-L-Carnitine/L-Carnitine, Arginine, Biotin, Betel nut, B-vitamins, Cacao, Cayenne, Chia seeds, Chlorella, Chlorophyll, Chromium picolinate, Coconut oil, Coenzyme Q-10, Cordyceps, Damiana, Dehydroepiandrosterone (DHEA), Eluthero, Ephedra, Fo-Ti, Free form amino acids, Ginkgo, Ginseng, which may be Asian or Red, Gotu kola/kula, Grape seed, Green coffee, Guayusa, Holy basil extract, Hoodia, Indian ginseng, Iodine, L-Theanine, L-Tyrosine, Maca, Mucuna pruriens, Niacin, Rhodiola rosea, Sage, Selenium, St. John's Wort, Taurine, Tea (Cammillia sinensis), Theobroma cacao, Tobacco, Xanthine, or Yohimbe.

b. Relaxant

The relaxant may comprise a natural relaxant, which may comprise a chamomile extract, lavender extract, lemon balm extract, or passion flower extract, or a combination thereof. While the relaxant may comprise a chamomile extract in particular, the relaxant may also comprise, or comprise an extract of, Ashwagandha root, Benzoin Resin, Bergamot Oil, Boswellia (Frankincense), Calamus root, Calcium, California poppy, Camphor, Catnip, Cedarwood, Celandine, Chaste tree/vitex/chasteberry, Cherries/cherry juice, Clary Sage, Coriander, Cornflowers, Corydalis, DHEA, Diphenhydramine, Dong quai, Fumitory, Geranium, Hemp Oil, Hyssop, Inositol, Jamaican Dogwood, Jasmine Flower Oil, Juniper Berries, Linden, Lotus, Magnesium, Marjoram, Melatonin, Milk peptides, Motherwort, Myrrh, Nard Oil, Niacinamide, Orange, Patchouli, Periwinkle, Potassium, Red clover, Rose, Sandalwood, Schisandra, Skullcap, Stevia herb, Sweet Woodruff, Tryptophan, Wild Cherry Bark, Wild lettuce, Wintergreen, or Ylang-Ylang.

c. Sedative

The sedative may comprise melatonin, which may comprise a melatonin from a natural source. The sedative may also be a valerian root extract, a hops extract, or a kava extract, or a combination thereof.

d. Nootropic

The nootropic may comprise a natural or synthetic nootropic, which may comprise niacinamise, pyridoxal 5 phosphate, methyltetrahydrofolate, methylcobalmin, calcium d-pantothenate, alpha-lipoic acid, L-tyrosine, L-glutamine, white willow bark powder, DMAE bitartrate, acetyl L-carnitine, or ginkgo leaves powder, or a combination thereof.

e. Pill Formation

The inner portion may comprise a capsule, and the outer portion may comprise a capsule coating. The capsule may be a softgel or a hard-shell capsule. The capsule may comprise a liquid fill, a powder fill, or a semi-solid fill; and a shell.

1. Liquid Fill

The liquid fill may comprise the stimulant, a liquid vehicle, a plasticizer, a surfactant, water, a solubilizing agent, and a suspending agent.

a. Liquid Vehicle

The liquid vehicle may comprise a lipophilic liquid or a semi-solid. The lipophilic liquid may comprise a vegetable oil and/or polyether. The semi-solid may comprise a hydrogenated oil, such as castor oil, and/or a wax such as bees wax.

b. Plasticizer

The plasticizer may comprise glycerol, glycerin, sorbitol, or propylene glycol, or a combination thereof.

c. Surfactant

The surfactant may comprise a lecithin, sorbitol, polysorbate, or sorbitan, or a combination thereof.

d. Solubilizing Agent

The solubilizing agent may comprise a beeswax or a mono-, di-, or triglyceride, or a combination thereof.

e. Suspending Agent

The suspending agent may comprise maltodextrin, sodium alginate, or xanthan gum, or a combination thereof.

2. Powder Fill

The powder fill may comprise the stimulant, a diluent, an anti-caking agent, and a lubricant.

a. Diluent

The diluent may comprise dicalcium phosphate, lactose, maltodextrin, microcrystalline cellulose, or a starch, or a combination thereof.

b. Anti-Caking Agent

The anti-caking agent may comprise magnesium silicate, silica gel, or talc, or a combination thereof.

c. Lubricant

The lubricant may comprise hydrogenated vegetable oil, magnesium stearate, mineral oil, or stearic acid, or a combination thereof.

3. Semi-Solid Fill

The semi-solid fill may comprise the stimulant, a semi-solid vehicle, a surfactant, and an emulsifying agent.

a. Semi-Solid Vehicle

The semi-solid vehicle may comprise hydrogenated palm oil, hydrogenated castor oil, cetyl alcohol, cetosteryl alcohol, a stearoyl polyoxylglyceride, a laurolyl polyoxyglyceride, or a combination thereof.

b. Surfactant

The surfactant may be a lecithin, sorbitol, polysorbate, or sorbitan, or a combination thereof.

c. Emulsifying Agent

The emulsifying agent may be polyethylene glycol, or poloxamer, or a combination thereof.

4. Shell

The shell may comprise a gelling agent, the plasticizer, an opacifier, a colorant, and water.

a. Gelling Agent

The gelling agent may comprise gelatin, a plant polysaccharide, a carrageenan, a modified starch, a cellulose or derivative thereof, or a combination of the foregoing. The modified starch may comprise starch hydrolysate. The cellulose derivative may comprise hypromellose or methylcellulose, or a combination thereof.

b. Opacifier

The opacifier may comprise titanium dioxide.

c. Plasticizer

The plasticizer may comprise glycerol, glycerin, sorbitol, or propylene glycol, or a combination thereof.

d. Outer Portion or Second Pill

The first pill or outer portion of a pill may comprise a coating, which may comprise the sedative and/or relaxant.

(1) Coating

The coating may comprise the sedative and/or the relaxant, a cellulose or derivative thereof, a polyether, a mineral oil, water, a plant resin or protein, and a surfactant. The polyether may comprise a polyethylene glycol, which may comprise a low molecular weight polyethylene glycol such as a PEG 300-600 or PEG 400, or a high molecular weight polyethylene glycol such as a PEG 4000-10,000, or a combination thereof.

e. Doses

The formulation may comprise 50-1000 mg of the stimulant, and more specifically may comprise 200-800, 300-600, 50, 100, 150, 200, 250, 300, 350, 400, 450, 500, 550, 600, 650, 700, 750, 800, 850, 900, 950, or 1000 mg of the stimulant. The formulation may also comprise 0.1-10 mg of the sedative, and more specifically may comprise 0.5-1.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mg of the sedative. The formulation may also comprise 0.1-10 mg of the relaxant, and more specifically may comprise 0.5-1.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mg of the relaxant.

If the formulation is a capsule, the capsule may comprise the ingredients listed in Tables 1 and

EXEMPLARY TABLE 1

| Ingredient | Amount (mg) |
|---|---|
| Green tea extract | 100-1000 |
| Vegetable oil | 100-1000 |
| Gelling agent | 10-1000 |
| Plasticizer | 1-500 |
| Opacifier | 0.1-50 |
| Water | 0.1-50 |
| Colorant | 0-50 |

EXEMPLARY TABLE 2
(Table: ‘Redbud Delayed Energy Bulk Capsule’)

| Ingredients | Label Claim | |
|---|---|---|
| NIACINAMIDE (98%), FREE FLOWING | 10.00 | mg |
| PYRIDOXAL 5 PHOSPHATE | 12.50 | mg |
| METHYLTETRAHYDROFOLATE | 200.00 | μg |
| METHYLCOBALAMIN 1% FOOD GRADE | 500.00 | μg |
| CALCIUM D-PANTOTHENATE (92%) | 20.00 | mg |
| ALPHA-LIPOIC ACID | 15.00 | mg |
| CAFFEINE ANHYDROUS (USP) | 120.00 | mg |
| PROPRIETARY BLEND | 500.00 | mg |
| GUARANA SEED 22% (CAFFEINE) | 170.00 | mg |
| L-GLUTAMINE FINE PWD | 125.00 | mg |
| L-TYROSINE | 75.00 | mg |
| WHITE WILLOW BARK PWD | 55.00 | mg |
| DMAE BITARTRATE NAT GRADE | 50.00 | mg |
| GINKGO LEAVES PWD | 25.00 | mg |
| EXCIPIENTS QS | | |
| LONG GRAIN RICE FLOUR | | |
| MAGNESIUM STEARATE VEG NF | | |
| SIPERNAT 50S | | |
| | Net Weight: | |
| Artificial RED color VCAPS PLUS SIZE 0 30 min | | |
| DELAYED RELEASE | | |

And the second pill or, in a single pill formulation the coating, may comprise the ingredients listed in Tables 3 and 4.

EXEMPLARY TABLE 3

| Ingredient | Amount (mg) |
|---|---|
| Cellulose derivative | 10-200 |
| Polyethylene glycol | 1-100 |
| Mineral oil | 0.1-10 |
| Sedative | 0.1-50 |
| Relaxant | 0.1-50 |

EXEMPLARY TABLE 4
(Table: ‘Redbud Sleep Chewable Bulk Tablet’)

| Ingredients | Label Claim | |
|---|---|---|
| Proprietary Blend | 200.0 | mg |
| GAMMA AMINOBUTYRIC ACID (GABA) | 100.0 | mg |
| MCT 40% PWD ON ACACIA HIGH FLOW 50% | 84.0 | mg |
| LEMON BALM LEAVES PWD | 10.0 | mg |
| LAVENDER FLOWER PWD | 1.0 | mg |
| VALERIAN ROOT EXT 4:1 | 1.0 | mg |
| L-TRYPTOPHAN GRAN | 1.0 | mg |
| HOPS FLOWER PWD | 1.0 | mg |
| PASSION FLOWER PWD | 1.0 | mg |
| CHAMOMILE FLOWER PE 4:1 | 1.0 | mg |
| Vitamins/Minerals | | |
| MAGNESIUM CITRATE 30% (MGCI304001) | 25.000 | mg |
| Other Ingredients | | |
| SORBITOL GRAN 18-75 MESH | | |
| PEPPERMINT FLAVOR NAT PWD #2005403 | | |
| LUO HAN GUO EXTRACT 50% MOGROSIDE V | | |
| STEARIC ACID NF PWD (TRISTAR NF) | | |
| MAGNESIUM STEARATE VEG NF | | |
| Ultramarine Blue E-5100 | | |
| SIPERNAT 50S | | |

5. Method of Nap Promotion

Provided herein is a method of promoting a nap in a subject. The nap may not induce grogginess. The method may comprise administering a formulation described herein to a subject in need thereof either as one pill or multiple pills

The present invention has multiple aspects, illustrated by the following non-limiting examples.

### Example 1

### Oral Capsule for Promoting Naps

This example describes a formulation of a single pill of Tables 5 and 6 or a coated capsule for promoting naps. The capsule contains the ingredients listed in Table 5 and 6.

EXEMPLARY TABLE 5

| Ingredient | Amount (mg) |
|---|---|
| Green tea extract | 500 |
| Vegetable oil | 300 |
| Gelatin | 265 |
| Glycerol | 50 |
| Titanium dioxide | 10 |
| Purified water | 6 |
| Coloring | 1 |

EXEMPLARY TABLE 6
(Table: ‘Redbud 90 Napjitsu Tincture Pineapple Peach Mango’)

| | Label Claim | |
|---|---|---|
| Ingredients | (per serving) | |
| PROPRIETARY OIL BLEND | 1831 | |
| FRACTIONATED COCONUT OIL (MCT OIL) | 1801.08 | mg |
| Grapefruit Pink 50-6140-02 | 13.00 | mg |
| ORANGE SWEET OIL 50-6200-01 | 12.00 | mg |
| Valerian 50-4739-01 oil | 1.00 | mg |
| Rosemary Tunisian 50-6230-02 | 1.00 | mg |
| LAV Bulgarian 50-6155-04 Lavender Bulgarian oil | 1.00 | mg |
| Chamomile Roman 50-6077-04 | 1.00 | mg |
| Lemon Argentina 50-6160-15 | 1.00 | mg |
| Copaiba 50-4237-01 | 0.01 | mg |
| PROPRIETARY BLEND | 20.00 | |
| GAMMA AMINOBUTYRIC ACID (GABA) | 7.00 | mg |
| L-THEANINE | 7.00 | mg |
| L-TYROSINE | 3.00 | mg |
| ACETYL L-CARNITINE HCL | 3.00 | mg |
| Other Ingredients | | |
| LUO HAN GUO EXTRACT 50% MOGROSIDE V | | |
| ORANGE PINEAPPLE NAT WONF FLAVOR 5418 PDR | | |
| ALL NAT PEACH TYPE FL #28722 JE45 | | |
| ALL NAT MANGO TYPE FL #28724 SZ76 | | |

The second pill contains the material of Table 7 or a coating for the pill of Tables 5 or 6, said pill or coating contains the ingredients listed in Table 7.

TABLE 7
Coating ingredients

| Ingredient | Amount (mg) |
|---|---|
| Hypromellose | 52 |
| Polyethylene glycol 400 | 28 |
| Mineral oil | 6 |
| Melatonin | 1 |
| Chamomile extract | 1 |

The formulation is made by mixing green tea extract, vegetable oil, glycerol, and purified water, with heating as necessary, to form a liquid fill solution or uniform suspension. The gelatin is heated to obtain the proper viscosity, and then the titanium oxide and coloring agents are added to form the capsule shell. Additional water is added as needed to obtain the desired viscosity for the shell. The gelatin is then formed into ribbons on encapsulation equipment, and the liquid is encapsulated within the gelatin using Oval No. 12 dies. The resulting softgel capsules are dried at the appropriate temperature and airflow.

The second pill or coating ingredients from Table 7 are mixed with water and heated as necessary to form a solution. As a coating, the solution is applied to the softgel capsules using a 60″ coating pan operated at the appropriate airflow, input temperature, and exhaust temperature.

### Example 2 Core Tablet

A core tablet comprising guarana extract, lactose, microcrystalline cellulose, sodium starch glycolate, hypromellose, and magnesium stearate, as shown in Table 8, was tested for its caffeine dissolution profile. The results are shown in Table 9.

TABLE 8
Tablet ingredients

| Ingredient | Amount (mg) |
|---|---|
| Guarana extract | 250 |
| Lactose | 245 |
| Microcrystalline cellulose | 476 |
| Sodium starch glycolate | 15 |
| Hypromello se | 10 |
| Magnesium stearate | 4 |

TABLE 9
Tablet dissolution

| Time (hr) | % dissolution |
|---|---|
| 1 | 34.1 |
| 2 | 50.3 |
| 3 | 67.0 |

Because less than 90% of the caffeine was dissolved after 2 hours, this core tablet formulation is not well-suited for use in promoting non-grogginess-inducing naps of short duration.

### Example 3 Core Tablet

A core tablet comprising guarana extract, calcium sulfate, croscarmellose sodium, magnesium stearate, microcrystalline cellulose, polyvinyl pyrrolidone, and sodium starch glycolate, as shown in Table 10, was tested for its caffeine dissolution profile. The results are shown in Table 11.

TABLE 10
Tablet ingredients

| Ingredient | Amount (mg) |
|---|---|
| Guarana extract | 250 |
| Calcium sulfate | 200 |
| Croscarmellose sodium | 25 |
| Magnesium stearate | 1 |
| Microcrystalline cellulose | 504 |
| Polyvinyl pyrrolidone | 10 |
| Sodium starch glycolate | 10 |

TABLE 11
Tablet dissolution

| Time (hr) | % dissolution |
|---|---|
| 0.5 | 31.0 |
| 1 | 57.5 |
| 2 | 87.5 |

Because less than 90% of the caffeine was dissolved after 1 hour, the core tablet formulation described is not well-suited in promoting short non-grogginess-inducing naps.

### Example 4

Each participant used a separate relaxing pill. All participants received the relaxing pill and the Nap 30 nootropic pill. Half of all participants were randomly assigned to a comparison group (coffee+placebo pill), while the other half were randomly assigned to a pure placebo group (placebo pill only). The Nap30 relaxant pill contained as described herein. The full study design is shown in the table below.

| | Day 1 | Day 2 |
|---|---|---|
| Arm 1 20 Participants | Blue pill + Nap 30 pill | 10 Placebo pill (Zeebo) + no coffee |
| | | 10 Placebo + coffee |
| Arm 2 20 Participants | 10 Placebo pill (Zeebo) only | Blue pill + Nap 30 pill |
| | 10 Placebo pill + coffee | |

Participants were asked to abstain from coffee for 6 hours prior to the anticipated time of their nap on both study days. Comparison and placebo arms were not instructed to nap on that day.

### Results

Nap 30 performed well across 7 of 8 metrics with self-reported improvement ranging from a low of 8% for mood to a whopping 56% for overall good feelings—defined as the combination of energy, focus, and sharpness.

### IMPROVED . . .

Overall, feeling good (energy, focus, sharpness) increased by 56%
Energy increased by 27%
Memory increased by 21%
Sharpness increased by 16%
Tiredness was reduced by 13%
Focus increased by 13% and 17% at its peak (3 hours later)
Mood increased by 8%

Compared with the Comparative arm (coffee+placebo) . . .

Nap 30 arm was superior for all of the metrics except for a tie for Mood at 8%.
Below is a comparison of percent change for Nap 30 and the Coffee Comparison arm from baseline to 5 hour follow-up showing unexpected superiority of the formulation:
Overall good feeling (energy, focus, sharpness): Nap 30 improved 56%|Coffee arm worsened −2%
Energy: Nap 30 improved 27%|Coffee arm improved 13%
Memory: Nap 30 improved 21%|Coffee arm improved 5%
Sharpness: Nap 30 improved 16%|Coffee arm improved 82%
Tiredness: Nap 30 improved 13%|Coffee arm worsened 15%
Focus: Nap 30 improved 13%|Coffee arm worsened 2%
Mood: Nap 30 improved 8%|Coffee arm tied at 8% improved

In general, Nap 30 respondents were significantly more likely to report a steady increase across seven metrics (mean comparisons), peaking at 5 hours—with far greater consistency than the comparison and the placebo arms. Results underscore the nootropic value of Nap 30. In effect, compared with coffee, Nap 30 gives staying power. Asked how satisfied they were with their experience with Nap 30, 92% reported feeling “satisfied”, while a total of 46% of respondents reported feeling “extremely satisfied”. Likelihood of recommendation reached 85% with 39% “extremely likely” to recommended to friends or family Over 90% (92%) of participants said they were likely to purchase with 24% “extremely likely”>. Asked which of 13 possible reasons participants would use Nap 30, “sleeping poorly” and “feeling tired” were affirmed by a majority (over 50%), with “need energy” being affirmed by 46%.

Taken only once during the 2-day study period, Nap 30 performed very well with respondents indicating a positive gain across all metrics at one or more time-points.

Data collected over 4 post-baseline intervals (30 minutes to 5 hours post-pill) found the greatest gains were realized at the 5-hour mark in most cases. By comparison, the coffee arm performed next best, but failed to achieve the long-lasting effects of Nap 30; particularly at the 5-hour mark.

Asked about their experience with Nap 30, participants reports were most favorable with a majority satisfied, likely to recommend to friends/family, and likely to purchase. While the foregoing disclosure discusses illustrative embodiments, it should be noted that various changes and modifications could be made herein without departing from the scope of the described aspects and/or embodiments as defined by the appended claims. Furthermore, although elements of the described aspects and/or embodiments may be described or claimed in the singular, the plural is contemplated unless limitation to the singular is explicitly stated. Additionally, all or a portion of any embodiment may be utilized with all or a portion of any other embodiment, unless stated otherwise.

While certain exemplary embodiments have been described and shown in the accompanying specification, it is to be understood that such embodiments are merely illustrative of and not restrictive on the broad invention, and that this invention is not be limited to the specific constructions and arrangements shown and described, since various other changes, combinations, omissions, modifications and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

1. A nap improving composition comprising a pill with a stimulant and a separate pill with a relaxant or sedative.

2. A nap improving oral formulation comprising an outer layer comprising a sedative and optionally a relaxant, and an inner layer comprising a stimulant.

3. The formulation of claim 2, wherein the inner layer comprises a capsule and the outer layer comprises a coating.

4. A method of promoting napping in a subject, comprising administering the oral formulation of claim 1 to a subject in need thereof.
